(19) United States
(12) Patent Application Publication
(10) Pub. No.: US 2026/0183456 A1
(43) Pub. Date: Jul. 2, 2026

(54) APPARATUSES AND METHODS FOR TREATING OPHTHALMIC DISEASES AND DISORDERS

(71) Applicant: BioTissue Holdings Inc., Miami, FL (US)

(72) Inventors: Scheffer TSENG, Pinecrest, FL (US); Ek Kia TAN, Miami, FL (US)

(21) Appl. No.: 19/400,523

(22) Filed: Nov. 25, 2025

Related U.S. Application Data
(63) Continuation of application No. 18/100,696, filed on Jan. 24, 2023, now Pat. No. 12,508,349.
Application No. 18/100,696 is a continuation of application No. 16/591,219, filed on Oct. 2, 2019, now Pat. No. 11,590,265.
Application No. 16/591,219 is a continuation of application No. 15/051,477, filed on Feb. 23, 2016, now abandoned.
(60) Provisional application No. 62/119,544, filed on Feb. 23, 2015.

(51) Int. Cl. A61L 31/00; A61F 9/00; A61L 31/14; A61L 31/16; A61N 1/04; A61N 1/36

(52) U.S. Cl. CPC A61L 31/005; A61F 9/0017; A61L 31/146; A61L 31/16; A61L 2300/406; A61L 2430/16; A61N 1/0452; A61N 1/36003

(57) ABSTRACT

The invention relates generally to the fields of biology and health sciences. More particularly, the invention relates to compositions and methods for modulating cellular physiology and pathological processing using a combination of compounds that can be found in amniotic membrane tissue and umbilical cord tissue preparations.

### CROSS-REFERENCE TO RELATED APPLICATIONS

This application is a continuation of U.S. application Ser. No. 18/100,696, filed on Jan. 24, 2023, which is a continuation of U.S. application Ser. No. 16/591,219, filed on Oct. 2, 2019, now U.S. Pat. No. 11,590,265, which is a continuation of U.S. application Ser. No. 15/051,477, filed on Feb. 23, 2016, which claims benefit of U.S. Provisional Application No. 62/119,544, filed Feb. 23, 2015, entitled “Apparatuses and Methods For Treating Ophthalmic Diseases and Disorders,” the contents of each are incorporated herein by reference in their entireties.

### FIELD OF THE INVENTION

The invention relates generally to the fields of biology and health sciences. More particularly, the invention relates to compositions and methods for modulating cellular physiology and pathological processing using a combination of compounds that can be found in amniotic membrane tissue and umbilical cord tissue preparations.

### BACKGROUND

With each blink of the eyelids, tears are spread across the front surface of the eye, known as the cornea. Tears provide lubrication, reduce the risk of eye infection, wash away foreign matter in the eye, and keep the surface of the eyes smooth and clear. Excess tears in the eyes flow into small drainage ducts, in the inner corners of the eyelids, which drain in the back of the nose.

Tears are produced by several glands in and around the eyelids. Tear production tends to diminish with age, with various medical conditions, or as a side effect of certain medicines. Environmental conditions such as wind and dry climates can also affect tear volume by increasing tear evaporation. When the normal amount of tear production decreases or tears evaporate too quickly from the eyes, symptoms of dry eye can develop.

Tears are made up of three layers: oil, water, and mucus. Each component serves a function in protecting and nourishing the front surface of the eye. A smooth oil layer helps to prevent evaporation of the water layer, while the mucin layer functions in spreading the tears evenly over the surface of the eye. If the tears evaporate too quickly or do not spread evenly over the cornea due to deficiencies with any of the three tear layers, dry eye symptoms can develop.

The most common form of dry eyes is due to an inadequate amount of the water layer of tears. This condition, called keratoconjunctivitis sicca (KCS), is also referred to as dry eye syndrome.

People with dry eyes may experience symptoms of irritated, gritty, scratchy, or burning eyes, a feeling of something in their eyes, excess watering, and blurred vision due to nerve loss or nerve damage in the cornea. Advanced dry eyes may damage the front surface of the eye and impair vision.

Traditionally ocular surface reconstruction has been used to treat acute chemical and thermal burns of the eye. This form of ocular surface reconstruction involves suturing a protective and/or therapeutic covering over the surface of the eye.

A variety of ophthalmic and non-ophthalmic conditions necessitate administration of various drugs to the eye. Eye drops and gels can be effective drug delivery vehicles, but can also have significant disadvantages. Specifically, eye drops mix with fluid in the tear film, but may have a residence time of only 2-5 minutes in the tear film. As little as 5% of the drug may be absorbed locally; some or all of the rest being carried from the lacrimal sac into the lacrimal duct, which can have potentially undesirable effects. Consequently, most of the drug may be wasted with less than ideal amounts delivered to the targeted tissue. Also, the presence of the drug in the bloodstream may have potentially harmful side effects. Gels may adhere more effectively to the eye, but can also blur the patient's vision. Both eye drops and gels may need to be reapplied frequently for some therapies, and patients may not administer the eye drops or gels as frequently as directed in at least some instances, such that the amount of drug delivered can be less than ideal. For example, in at least some instances a substantial number of patients may not refill their prescription after one year, and the substantial number of patients can be up to fifty percent in some instances.

Current treatments for dry eyes aim to restore or maintain the normal amount of tears in the eye to minimize dryness and related discomfort and to maintain eye health while current treatments for acute chemical and thermal burns require the use of sutures. What is needed is an apparatus that can be inserted into the eye of a patient that will induce blinking and may optionally deliver one or more therapeutic agents to the eye of the patient and further may optionally cover the surface of the eye with a therapeutic agent.

### SUMMARY

In one embodiment the current application describes an apparatus for treating dry eye or other diseases or disorders of the eye, by insertion of the device onto the surface of the eye of a patient in need thereof, wherein the apparatus comprises: one or more flexible tubular structures, wherein the one or more flexible tubular structures comprise a material selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); polyurethane (PU); umbilical cord tissue; amniotic membrane tissue; placental tissue; chorion tissue and any combination thereof.

In one embodiment the current application describes an apparatus for treating dry eye or other diseases or disorders of the eye, by insertion of the apparatus onto the surface of the eye of a patient in need thereof, wherein the apparatus comprises: one or more flexible tubular structures, wherein the one or more flexible tubular structures comprise a material selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); polyurethane (PU); umbilical cord tissue; amniotic membrane tissue; placental tissue; chorion tissue and any combination thereof; and one or more flexible fastening cuffs, wherein the inner surface of the one or more flexible fastening cuffs are frictionally engaging the outer surface of the one or more flexible tubular structures.

In one embodiment the current application describes an apparatus for treating dry eye or other diseases or disorders of the eye, by insertion of the device onto the surface of the eye of a patient in need thereof, wherein the apparatus comprises: at least one sheet comprising a material selected from the group consisting of: umbilical cord tissue; amniotic membrane tissue; placental tissue; chorion tissue; ethylene vinyl acetate (EVA), polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof; and at least one tubular structure comprising a second material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof, wherein at least one sheet and at least one tubular structure are adhering to maintain physical contact with one another.

In one embodiment the current application describes an apparatus for treating dry eye or other diseases or disorders of the eye, by insertion of the device onto the surface of the eye of a patient in need thereof, wherein the apparatus comprises: at least one first sheet comprising a material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof; and at least one second sheet comprising a second material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof, wherein the at least one first sheet and the at least one second sheet are adhering to maintain physical contact with one another.

In certain embodiments the current application describes the use of an apparatus according to the current application to promote an increase in tissue sensation, to induce a patient to blink and tear more frequently to prevent dry eye, to promote nerve growth, promote nerve regeneration or a combination thereof in a contacted tissue, to reduce an inflammatory response in a contacted tissue, to increase Tear Breakup Time in a patient suffering from dry eye disease, to increase tear osmolarity in a patient suffering from dry eye disease, to decrease corneal straining in a patient suffering from dry eye disease, to increase the score on Schirmer's test in a patient suffering from dry eye disease or combinations thereof.

### BRIEF DESCRIPTION OF THE FIGURES

FIG. 1—Illustrates an apparatus of the present application, wherein the apparatus comprises: one or more flexible tubular structures having at least one porous inlet and at least one porous outlet.

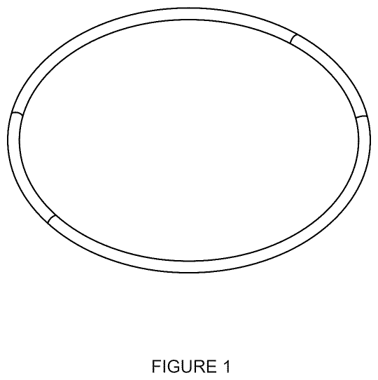

FIG. 2—Illustrates an apparatus of the present application, wherein the apparatus comprises: one or more flexible tubular structures having at least one porous inlet and at least one porous outlet; and one or more flexible fastening cuffs, wherein the inner surface of the one or more flexible fastening cuffs are frictionally engaging the outer surface of the one or more flexible tubular structures.

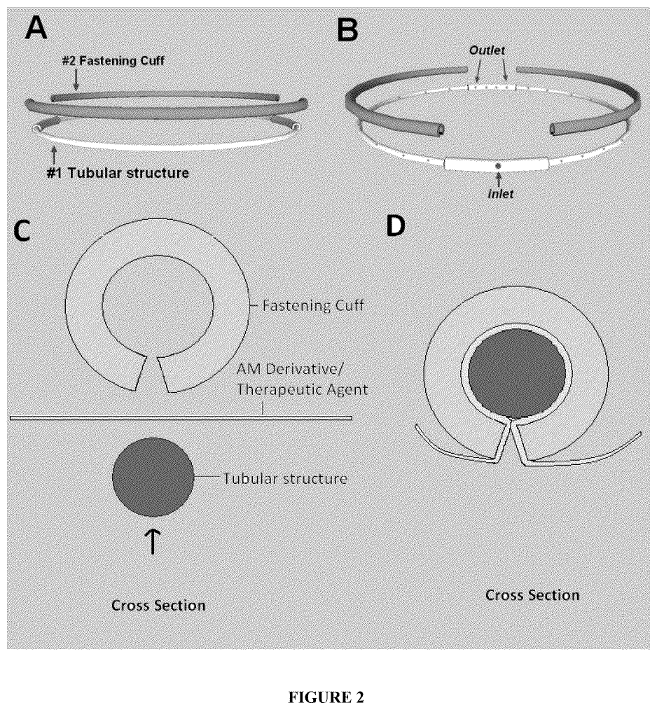

FIG. 3—Illustrates an apparatus of the present application, wherein the apparatus comprises: one or more flexible tubular structures having at least one porous inlet and at least one porous outlet; and one or more flexible fastening cuffs, wherein the opening defined by the outer circumference of the one or more flexible tubular structures is covered by a sheet frictionally engaged between the one or more flexible fastening cuffs and the flexible tubular structure.

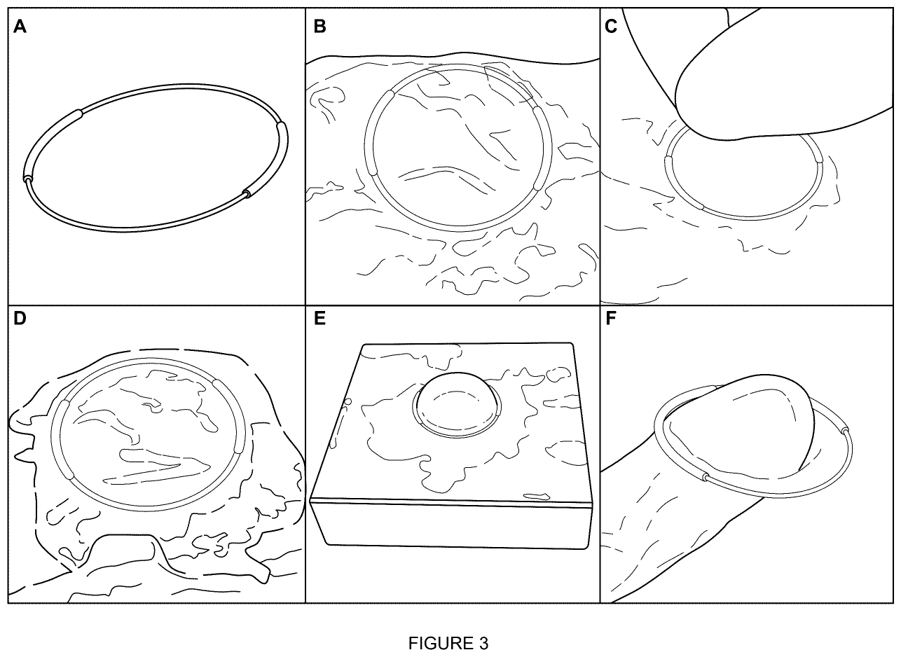

FIG. 4—Illustrates an apparatus of the present application, wherein the apparatus comprises: one or more flexible tubular structures having at least one porous inlet and at least one porous outlet, wherein the opening defined by the outer circumference of the one or more flexible tubular structures is covered by a sheet frictionally engaged on the flexible tubular structure.

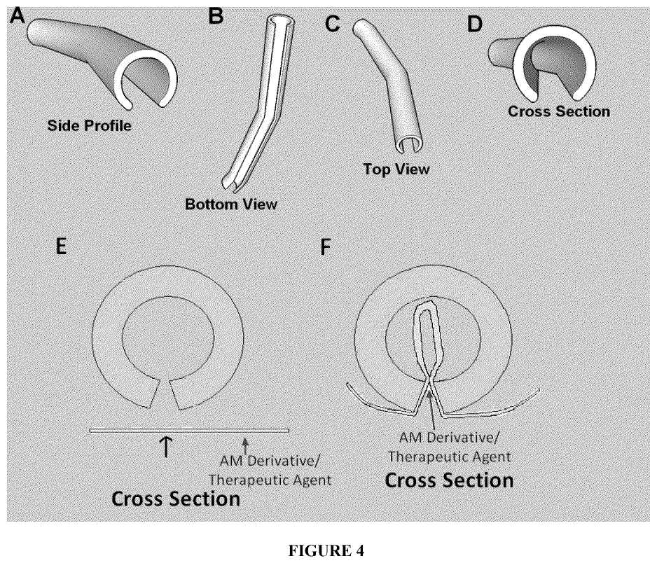

FIG. 5—Illustrates and apparatus of the present application, wherein the apparatuus comprises: at least one sheet of a therapeutic agent and at least one tubular structure of a polymeric material, wherein the tubular structure is formed upon and adhered to the sheet of the therapeutic agent.

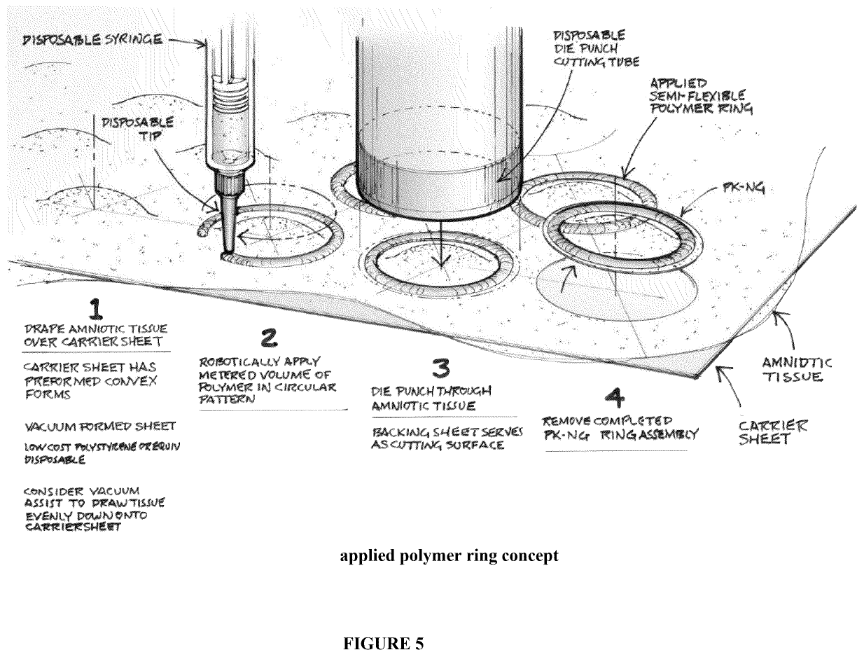

### DETAILED DESCRIPTION

A description of preferred embodiments of the current application follows. It will be understood that the particular embodiments of the application are shown by way of illustration and not as limitations of the application. At the outset, the embodiments are described in their broadest overall aspects, with a more detailed description following. The features and other details of the apparatuses and uses of the embodiments will be further pointed out in the claims.

The present application is directed to apparatuses and uses of the apparatuses for treating dry eye or other diseases or disorders of the eye.

In a first embodiment the current application describes an apparatus for treating dry eye or other diseases or disorders of the eye, by insertion of the device onto the surface of the eye of a patient in need thereof, wherein the apparatus comprises: one or more flexible tubular structures, wherein the one or more flexible tubular structures comprise a material selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); polyurethane (PU); umbilical cord tissue; amniotic membrane tissue; placental tissue; chorion tissue and any combination thereof. In some embodiments the apparatus is configured to reside in at least one of the group consisting of: superior conjunctiva fornix, inferior conjunctiva fornix and combinations thereof. While in other embodiments the apparatus is configured to reside in both conjunctiva fornices. In certain embodiments the apparatus deepen the fornix tear reservoir by stretching the conjunctival sac. In some embodiments the apparatus induces blinking.

In other embodiments the one or more flexible tubular structures have a length of about 0.5 cm to about 15 cm, a circumference of about 2 cm to about 15 cm or a circumference of about 4 cm to about 12 cm, an outside diameter of about 0.5 cm to about 5.0 cm or an outside diameter of about 1.2 cm to about 3.5 cm, a cross sectional diameter of about 0.01 cm to about 1.0 cm or a cross sectional diameter of about 0.05 cm to about 0.2 cm.

In certain embodiments the one or more flexible tubular structures the one or more flexible tubular structures contain at least one porous inlet and at least one porous outlet and the at least one porous inlet and the at least one porous outlet taken together form an osmotic pump when in contact with tears in the patient's eye.

In further embodiments the apparatus is configured to deliver one or more therapeutic agents to the patient, the therapeutic agent is delivered by diffusion through the net movement of solute from an area of high concentration to an area of low concentration, the therapeutic agent is delivered through compression of the apparatus via blinking, the one or more therapeutic agents may be incorporated into the material of the one or more flexible tubular structures, on the surface of the one or more flexible tubular structures, inside the one or more flexible tubular structures or combinations thereof, the one or more flexible tubular structures are configured to dissolve when in contact with tears in the patient's eye, the one or more therapeutic agents may be frictionally engaged onto a surface of the one or more flexible tubular structures, into the one or more flexible tubular structures or a combination thereof, the one or more therapeutic agents may be one or more sheets covering from about 25% to about 100% of the opening defined by the outer circumference of the one or more flexible tubular structures, the one or more therapeutic agents is selected from the group consisting of: antibacterial antibiotics, synthetic antibacterials, antifungal antibiotics, synthetic antifungals, umbilical cord tissue, amniotic membrane tissue, placental tissue, chorion tissue, antineoplastic agents, steroidal anti-inflammatory agents, non-steroidal anti-inflammatory agents, anti-allergic agents, glaucoma-treating agents, antiviral agents, and anti-mycotic agents, wherein the one of more therapeutic agents may be dispersed in a polymer, wherein the polymer is selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); and polyurethane (PU), wherein the umbilical cord tissue, amniotic membrane tissue, placental tissue or chorion tissue may be present in any form from the group consisting of: lyophilized, fresh, sheet, morselized, powder matrix, extract, purified extract, previously frozen, suspension and on a physiologically acceptable support.

In some embodiments the apparatus further comprises a power source at least one positive electrode and at least one negative electrode wherein the apparatus is configured to introduce an electrical stimulus between about 0.1 milliamps and about 50 milliamps to the muscles of the patient's eyelids.

In a second embodiment the current application describes an apparatus for treating dry eye or other diseases or disorders of the eye, by insertion of the apparatus onto the surface of the eye of a patient in need thereof, wherein the apparatus comprises: one or more flexible tubular structures, wherein the one or more flexible tubular structures comprise a material selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); polyurethane (PU); umbilical cord tissue; amniotic membrane tissue; placental tissue; chorion tissue and any combination thereof; and one or more flexible fastening cuffs, wherein the inner surface of the one or more flexible fastening cuffs are frictionally engaging the outer surface of the one or more flexible tubular structures. In some embodiments the apparatus is configured to reside in at least one of the group consisting of: superior conjunctiva fornix, inferior conjunctiva fornix and combinations thereof. In other embodiments the apparatus is configured to reside in both conjunctiva fornices. In certain embodiments the apparatus deepen the fornix tear reservoir by stretching the conjunctival sac. In still other embodiments the apparatus induces blinking.

In some embodiments the one or more flexible tubular structures have a length of about 0.5 cm to about 15 cm, a circumference of about 2 cm to about 15 cm or a circumference of about 4 cm to about 12 cm, an outside diameter of about 0.5 cm to about 5.0 cm or an outside diameter of about 1.2 cm to about 3.5 cm, a cross sectional diameter of about 0.01 cm to about 1.0 cm or a cross sectional diameter of about 0.05 cm to about 0.2 cm.

In certain embodiments the one or more flexible tubular structures the one or more flexible tubular structures contains at least one porous inlet and at least one porous outlet and the at least one porous inlet and the at least one porous outlet taken together form an osmotic pump when in contact with tears in the patient's eye.

In some embodiments the apparatus is configured to deliver one or more therapeutic agents to the patient, the therapeutic agent is delivered by diffusion through the net movement of solute from an area of high concentration to an area of low concentration, the therapeutic agent is delivered through compression of the apparatus via blinking, the one or more therapeutic agents may be delivered from the one or more flexible fastening cuffs, the one or more flexible tubular structures or a combination thereof, the one or more therapeutic agents may be incorporated into the material of the one or more flexible fastening cuffs, on the surface of the one or more flexible fastening cuffs or combinations thereof, the one or more flexible fastening cuffs are configured to dissolve when in contact with tears in the patient's eye, the one or more therapeutic agents may be incorporated into the material of the flexible tubular structure, on the surface of the flexible tubular structure, inside the flexible tubular structure or combinations thereof, the one or more flexible tubular structures are configured to dissolve when in contact with tears in the patient's eye, the one or more therapeutic agents may be frictionally engaged between the one or more flexible fastening cuffs and the flexible tubular structure, the one or more therapeutic agents may be one or more sheets covering from about 25% to about 100% of the opening defined by the outer circumference of the flexible tubular structure, the one or more therapeutic agents is selected from the group consisting of: antibacterial antibiotics, synthetic antibacterials, antifungal antibiotics, synthetic antifungals, umbilical cord tissue, amniotic membrane tissue, placental tissue, chorion tissue, antineoplastic agents, steroidal anti-inflammatory agents, non-steroidal anti-inflammatory agents, anti-allergic agents, glaucoma-treating agents, antiviral agents, and anti-mycotic agents, wherein the one of more therapeutic agents may be dispersed in a polymer, wherein the polymer is selected from the group consisting of: ethylene vinyl acetate (EVA); polydimethylsiloxane (PDMS); polymethylmetacrylate (PMMA); polyethylene teraphthalate (PET); polycarbonate (PC); hydrogels; polyvinyl chloride (PVC); rubber; latex; polyethylene (PE); silicone; polytetrafluoroethylene (PTFE); and polyurethane (PU), wherein the umbilical cord tissue, amniotic membrane tissue, placental tissue or chorion tissue may be present in any form from the group consisting of: lyophilized, fresh, sheet, morselized, powder matrix, extract, purified extract, previously frozen, suspension and on a physiologically acceptable support.

In other embodiments the apparatus further comprises a power source at least one positive electrode and at least one negative electrode wherein the apparatus is configured to introduce an electrical stimulus between about 0.1 milliamps and about 50 milliamps to the muscles of the patient's eyelids.

In a third embodiment the current application describes an apparatus for treating dry eye or other diseases or disorders of the eye, by insertion of the device onto the surface of the eye of a patient in need thereof, wherein the apparatus comprises: at least one sheet comprising a material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof; and at least one tubular structure comprising a second material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof, wherein at least one sheet and at least one tubular structure are adhering to maintain physical contact with one another.

In some embodiments the apparatus is configured to reside in at least one of the group consisting of: superior conjunctiva fornix, inferior conjunctiva fornix and combinations thereof. In other embodiments the apparatus is configured to reside in both conjunctiva fornices. In certain embodiments the apparatus induces blinking. In still other embodiments the apparatus deepens the fornix tear reservoir by stretching the conjunctival sac.

In some embodiments the at least one tubular structure has a circumference of about 2 cm to about 15 cm, the at least one tubular structure has a circumference of about 4 cm to about 12 cm, the at least one tubular structure has an outside diameter of about 0.5 cm to about 5.0 cm or the at least one tubular structure has an outside diameter of about 1.2 cm to about 3.5 cm, the at least one tubular structure has a cross sectional diameter of about 0.01 cm to about 1.0 cm or the at least one tubular structure has a cross sectional diameter of about 0.05 cm to about 0.2 cm In other embodiments the apparatus is configured to deliver one or more additional therapeutic agents to the patient, the one or more additional therapeutic agents may be incorporated into the material of the at least one tubular structure, on the surface of the at least one tubular structure or combinations thereof, the at least one tubular structure is configured to dissolve when in contact with tears in the patient's eye, the one or more additional therapeutic agents is selected from the group consisting of: antibacterial antibiotics, synthetic antibacterials, antifungal antibiotics, synthetic antifungals, umbilical cord tissue, amniotic membrane tissue, placental tissue, chorion tissue, antineoplastic agents, steroidal anti-inflammatory agents, non-steroidal anti-inflammatory agents, anti-allergic agents, glaucoma-treating agents, antiviral agents, and anti-mycotic agents, the umbilical cord tissue, amniotic membrane tissue, placental tissue or chorion tissue may be present in any form from the group consisting of: lyophilized, fresh, sheet, morselized, powder matrix, extract, purified extract, previously frozen, suspension and on a physiologically acceptable support, the apparatus further comprises a power source at least one positive electrode and at least one negative electrode wherein the apparatus is configured to introduce an electrical stimulus between about 0.1 milliamps and about 50 milliamps to the muscles of the patient's eyelids.

In certain embodiments the at least one tubular structure is formed upon a surface of the at least one sheet by use of a three-dimensional (3D) printer or the at least one tubular structure is formed upon a surface of the at least one sheet by use of polymer extrusion means.

In a fourth embodiment the current application describes an apparatus for treating dry eye or other diseases or disorders of the eye, by insertion of the device onto the surface of the eye of a patient in need thereof, wherein the apparatus comprises: at least one first sheet comprising a material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof; and at least one second sheet comprising a second material selected from the group consisting of: umbilical cord tissue; amniotic membrane tissue; placental tissue; chorion tissue; ethylene vinyl acetate (EVA); polydimethylsiloxane (PDMS); polymethylmetacrylate (PMMA); polyethylene teraphthalate (PET); fibrins; polycarbonate (PC); hydrogels; polyvinyl chloride (PVC); rubber; latex; polyethylene (PE); silicone; polytetrafluoroethylene (PTFE); polyurethane (PU) and combinations thereof, wherein the at least one first sheet and the at least one second sheet are adhering to maintain physical contact with one another.

In some embodiments the apparatus is configured to reside in at least one of the group consisting of: superior conjunctiva fornix, inferior conjunctiva fornix and combinations thereof. In other embodiments the apparatus is configured to reside in both conjunctiva fornices. In certain embodiments the apparatus induces blinking. In further embodiments the apparatus deepens the fornix tear reservoir by stretching the conjunctival sac.

In some embodiments the apparatus has a circumference of about 2 cm to about 15 cm, the apparatus has a circumference of about 4 cm to about 12 cm, the apparatus has an outside diameter of about 0.5 cm to about 5.0 cm or the apparatus has an outside diameter of about 1.2 cm to about 3.5 cm.

In other embodiments the apparatus is configured to deliver one or more additional therapeutic agents to the patient, the one or more additional therapeutic agents may be incorporated into the material of the polymeric sheet, on the surface of the polymeric sheet or combinations thereof, the polymeric sheet is configured to dissolve when in contact with tears in the patient's eye, the one or more additional therapeutic agents is selected from the group consisting of: antibacterial antibiotics, synthetic antibacterials, antifungal antibiotics, synthetic antifungals, umbilical cord tissue, amniotic membrane tissue, placental tissue, chorion tissue, antineoplastic agents, steroidal anti-inflammatory agents, non-steroidal anti-inflammatory agents, anti-allergic agents, glaucoma-treating agents, antiviral agents, and anti-mycotic agents, the umbilical cord tissue, amniotic membrane tissue, placental tissue or chorion tissue may be present in any form from the group consisting of: lyophilized, fresh, sheet, morselized, powder matrix, extract, purified extract, previously frozen, suspension and on a physiologically acceptable support, the apparatus further comprises a power source at least one positive electrode and at least one negative electrode wherein the apparatus is configured to introduce an electrical stimulus between about 0.1 milliamps and about 50 milliamps to the muscles of the patient's eyelids.

In a fifth embodiment the present application describes the use of an apparatus of the application to promote an increase in tissue sensation, to induce a patient to blink and tear more frequently to prevent dry eye, to promote nerve growth, wherein the increase in nerve growth is between about 10% and about 100%, promote nerve regeneration, wherein the increase in nerve regeneration is between about 10% and about 100%, to reduce an inflammatory response in a contacted tissue, to increase Tear Breakup Time in a patient suffering from dry eye disease, to increase tear osmolarity in a patient suffering from dry eye disease, to decrease corneal straining in a patient suffering from dry eye disease, to increase the score on Schirmer's test in a patient suffering from dry eye disease or combinations thereof.

### Ophthalmic Formulations

Unless the intended purpose of use is affected adversely, the apparatuses of the present application may further comprise one or more additional therapeutically-active agents. Specific therapeutically-active agents include, but are not limited to: antibacterial antibiotics, synthetic antibacterials, antifungal antibiotics, synthetic antifungals, antineoplastic agents, steroidal anti-inflammatory agents, non-steroidal anti-inflammatory agents, tissue preparations, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself.

Examples of the antibacterial antibiotics include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), . beta. lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefmetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Examples of the synthetic antibacterials include, but are not limited to: 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, N₂-formylsulfisomidine, N₄-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, 4-sulfanilamidosalicylic acid, N₄-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

Examples of the antifungal antibiotics include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin).

Examples of the synthetic antifungals include, but are not limited to: allylamines (e.g., butenafine, naftifine, terbinafine), imidazoles (e.g., bifonazole, butoconazole, chlordantoin, chlormiidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Examples of the antineoplastic agents include, but are not limited to: antineoplastc antibiotics and analogs (e.g., aclacinomycins, actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites exemplified by folic acid analogs (e.g., denopterin, edatrexate, methotrexate, piritrexim, pteropterin, TOMUDEX®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur).

Examples of the steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the non-steroidal anti-inflammatory agents include, but are not limited to: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (erg., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., am piroxicam, droxicam, isoxicam, lornoxicam, piroxicam, tenoxicam), E-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Examples of tissue preparations include, but are not limited to: umbilical cord tissue, amniotic membrane tissue, placental tissue and chorion tissue wherein, the umbilical cord tissue, amniotic membrane tissue, placental tissue or chorion tissue may be present in any form from the group consisting of: lyophilized, fresh, sheet, morselized, powder matrix, extract, purified extract, previously frozen, suspension and on a physiologically acceptable support.

Examples of anti-allergic agents include, but are not limited to: tranilast, ketotifen fumarate, pheniramine, diphenhydramine hydrochloride, and sodium cromoglicate.

Examples of glaucoma-treating agents include, but are not limited to: pilocarpine hydrochloride, latanoprost, timolol, and isopropylunoprostone.

Examples of antiviral agents include, but are not limited to: idoxuridine, acyclovir, and trifluorouridine.

Examples of anti-mycotic agents include, but are not limited to: pimaricin, fluconazole, miconazole, amphotericin B, flucytosine, and itraconazole.

### Ophthalmic Combinations

Unless the intended purpose of use is affected adversely, the apparatus of the present application may be administered concurrently with one or more therapeutically-active agents. Specific therapeutically-active agents include, but are not limited to: antibacterial antibiotics, synthetic antibacterials, antifungal antibiotics, synthetic antifungals, antineoplastic agents, steroidal anti-inflammatory agents, non-steroidal anti-inflammatory agents, tissue preparations, anti-allergic agents, glaucoma-treating agents, antiviral agents, and anti-mycotic agents. Further contemplated are any derivatives of the therapeutically-active agents which may include, but not be limited to: analogs, salts, esters, amines, amides, alcohols and acids derived from an agent of the invention and may be used in place of an agent itself.

### Excipients

The ophthalmic formulations may contain various excipients incorporated ordinarily, such as buffering agents (e.g., phosphate buffers, borate buffers, citrate buffers, tartarate buffers, acetate buffers, amino acids, sodium acetate, sodium citrate and the like), isotonicity agents (e.g., saccharides such as sorbitol, glucose and mannitol, polyhydric alcohols such as glycerin, concentrated glycerin, polyethylene glycol and propylene glycol, salts such as sodium chloride), preservatives or antiseptics (e.g., benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like), solubilizing aids or stabilizing agents (e.g., cyclodextrins and their derivatives, water-soluble polymers such as polyvinyl pyrrolidone, or carbomer, surfactants such as polysorbate 80 (Tween 80)), pH modifiers (e.g., hydrochloric acid, acetic acid, phosphoric acid, sodium hydroxide, potassium hydroxide, ammonium hydroxide and the like), thickening agents (e.g., hydroxyethyl cellulose, hydroxypropyl cellulose, methyl cellulose, hydroxypropylmethyl cellulose, carboxymethyl cellulose and their salts), chelating agents (e.g., sodium edetate, sodium citrate, condensed sodium phosphate) and the like. Descriptions of compounds used in standard ophthalmic formulations may be found in, for example, Remington's Pharmaceutical Sciences, latest edition, Mack Publishing Co. Easton Pa.

Non-limiting examples of the contemplated excipients include a buffer, osmotic agent, demulcent, surfactant, emollient, tonicity agent, and/or a preservative component.

EXAMPLES

### Example 1

A circular flexible tubular structure having an inside diameter of 3.5 cm and an annular groove situated at its perimeter was placed on a surface with the annular groove facing upwards. A sheet of amniotic membrane tissue was placed over the circular flexible tubular structure and the sheet was forced into the annular groove such that the sheet and the flexible tubular structure became frictionally engaged with the sheet covering the opening defined by the inside diameter of the flexible tubular structure.

### Example 2

A circular flexible tubular structure having an inside diameter of 3.5 cm was placed on a surface. A sheet of amniotic membrane tissue was placed over the circular flexible tubular structure. Two fastening cuffs were placed over different segment of the flexible tubular structure thereby frictionally engaging the sheet between the fastening cuffs and the flexible tubular structure with the sheet covering the opening defined by the inside diameter of the flexible tubular structure.

Although the present disclosure has been described in considerable detail with reference to certain preferred versions thereof, other versions are possible. Therefore, the spirit and scope of the application should not be limited to the description of the preferred versions described herein.

Although compositions, materials, and methods similar or equivalent to those described herein can be used in the practice or testing of the present invention, suitable preparations, methods and materials are described herein. All publications mentioned herein are incorporated by reference in their entirety. In the case of conflict, the present specification, including definitions will control. In addition, the particular embodiments discussed below are illustrative only and not intended to be limiting.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

1. An ophthalmic device comprising a flexible tubular structure, wherein the flexible tubular structure is configured for insertion into a fornix of an eye, wherein the flexible tubular structure further comprises an amniotic membrane tissue, an umbilical cord tissue, a placental tissue, or a chorion tissue; and wherein the flexible tubular structure is configured to dissolve when in contact with tears, whereupon the amniotic membrane tissue, the umbilical cord tissue, the placental tissue, or the chorion tissue is released from the flexible tubular structure and contacts the surface of the eye.

2. The ophthalmic device of claim 1, wherein the flexible tubular structure comprises an amniotic membrane tissue or an umbilical cord tissue.

3. The ophthalmic device of claim 1, wherein the flexible tubular structure comprises a placental tissue or a chorion tissue.

4. The ophthalmic device of claim 1, wherein the fornix is a superior conjunctiva fornix or an inferior conjunctiva fornix.

5. The ophthalmic device of claim 1, wherein the flexible tubular structure further comprises at least one porous inlet and at least one porous outlet.

6. The ophthalmic device of claim 5, wherein the at least one porous inlet and the at least one porous outlet form an osmotic pump when in contact with tears in the eye.

7. The ophthalmic device of claim 1, wherein the amniotic membrane tissue, umbilical cord tissue, placental tissue or chorion tissue are fresh or previously frozen.

8. The ophthalmic device of claim 1, wherein the amniotic membrane tissue, umbilical cord tissue, placental tissue or chorion tissue are an extract, lyophilized, powdered, or morselized.

9. The ophthalmic device of claim 1, wherein any of the amniotic membrane tissue, umbilical cord tissue, placental tissue or chorion tissue is a sheet.

10. The ophthalmic device of claim 1, wherein the flexible tubular structure is annular in shape.

11. The ophthalmic device of claim 10, wherein the ophthalmic device further comprises an AM, UC, placental, or chorion tissue sheet attached thereto.

12. The ophthalmic device of claim 11, wherein the AM, UC, placental, or chorion tissue sheet are configured to contact the surface of the eye.

13. The ophthalmic device of claim 1, wherein the device induces blinking.

14. The ophthalmic device of claim 1, wherein the device comprises at least one additional therapeutically active agent.

15. The ophthalmic device of claim 14 wherein the least one additional therapeutically active agent comprises an antibacterial antibiotic, a synthetic antibacterial, an antifungal antibiotic, a synthetic antifungal, an antineoplastic agent, a steroidal anti-inflammatory agent, a non-steroidal anti-inflammatory agent, a tissue preparation, an anti-allergic agent, a glaucoma-treating agent, an antiviral agent, an anti-mycotic agent, or any combination thereof.

16. A method of treating a disease or disorder of an eye of an individual in need thereof, the method comprising administering the ophthalmic device of claim 1 to the eye of the individual in need thereof, thereby treating the disease or disorder of the eye.

17. The method of claim 16, wherein the device is administered concurrently with the at least one additional therapeutically active agent.

18. The method of claim 17, wherein the at least one additional therapeutically active agent comprises an antibacterial antibiotic, a synthetic antibacterial, an antifungal antibiotic, a synthetic antifungal, an antineoplastic agent, a steroidal anti-inflammatory agent, a non-steroidal anti-inflammatory agent, a tissue preparation, an anti-allergic agent, a glaucoma-treating agent, an antiviral agent, an anti-mycotic agent, or any combination thereof.

19. The method of claim 16, wherein the ophthalmic device (i) induces the individual to blink and tear more frequently to prevent dry eye, (ii) increases nerve growth, wherein the increase in nerve growth is between about 10% and about 100%, (iii) increases nerve regeneration, wherein the increase in nerve regeneration is between about 10% and about 100%, (iv) promotes an increase in tissue sensation, (v) reduces an inflammatory response in a contacted tissue, (vi) increases tear breakup time in an individual suffering from dry eye disease, (vii) increases tear osmolality in an individual suffering from dry eye disease, (viii) decreases corneal straining in an individual suffering from dry eye disease, (ix) increases the score on Schirmer's test in an individual suffering from dry eye disease, or combinations thereof.

20. The method of claim 16, wherein the disease or disorder of the eye of the individual comprises dry eye disease or other ophthalmic diseases or disorders.
